(19) United States
(12) Patent Application Publication
(10) Pub. No.: US 2025/0003981 A1
(43) Pub. Date: Jan. 2, 2025

(54) IMPROVED METHODS FOR DETECTING AND TREATING ENDOMETRIOSIS

(71) Applicant: The Feinstein Institutes for Medical Research, Manhasset, NY (US)

(72) Inventors: Christine Metz, Great Neck, NY (US); Peter K. Gregersen, Larchmont, NY (US)

(21) Appl. No.: 18/706,278

(22) PCT Filed: Dec. 5, 2022
(86) PCT No.: PCT/US22/51821

§ 371 Date: Apr. 30, 2024

Related U.S. Application Data
(60) Provisional application No. 63/308,281, filed on Feb. 9, 2022.
(60) Provisional application No. 63/286,705, filed on Dec. 7, 2021.

(51) Int. Cl. G01N 33/68; C12Q 1/37; C12Q 1/44; C12Q 1/6806; C12Q 1/686; C12Q 1/6883; G01N 1/40; G01N 15/10; G01N 15/14

(52) U.S. Cl. CPC G01N 33/6848; C12Q 1/37; C12Q 1/44; C12Q 1/6806; C12Q 1/686; C12Q 1/6883; G01N 1/4077; G01N 15/14; G01N 33/6893; C12Q 2600/158; G01N 2001/4088; G01N 2015/1006; G01N 2333/922; G01N 2333/96486; G01N 2800/364

(57) ABSTRACT

A method of non-invasively diagnosing endometriosis in a subject using single cell isolation methods and kits which separate somatic cells and epithelial tissues, with an additional step of disaggregating epithelial tissues after separation single somatic cells from a menstrual effluent sample, determining uterine NK cells, T-cells, and/or B-cells, along with diagnosing and treating dysmenorrhea.

### CROSS-REFERENCE TO RELATED APPLICATIONS

This application claims benefit of U.S. Provisional Application No. 63/286,705, filed Dec. 7, 2021 and U.S. Provisional Application No. 63/308,281, filed Feb. 9, 2022, the contents of each of which are hereby incorporated by reference.

### BACKGROUND OF THE INVENTION

The disclosures of all publications, patents, patent application publications and books referred to in this application are hereby incorporated by reference in their entirety into the subject application to more fully describe the art to which the subject invention pertains.

Endometriosis is a chronic and underdiagnosed disease which affects 5-10% of women of childbearing age and is characterized by growth of endometrial-like tissue outside of the uterus, most often in the peritoneal cavity. Delay in diagnosis is a major problem for management of this disorder, and treatment is often not initiated until the disease has progressed for many years. Currently in the US, diagnosis of endometriosis can take up to 7 to 10 years for an individual. Endometriosis is a very complex disease, sometimes with vague and non-specific symptoms. Many women present, for example, with GI symptoms and they end up seeing a gastroenterologist as part of their diagnostic journey. Thus, it is critically important to find methods and means for accurate and early diagnosis of endometriosis. In addition, dysmenorrhea in adolescents has both endometriosis-related and endometriosis-unrelated occurrences. To avoid unnecessary or incorrect intervention in this population, it is important to be able to distinguish between those who would benefit from endometriosis treatment and those for whom such treatment will be of no benefit or a burden.

Although the exact etiology of endometriosis remains unknown, retrograde menstruation is recognized as a common underlying factor leading to the deposit of menstrual effluent (ME) into the peritoneal cavity. Differences in the cellular biology and genetics of the cells within ME are therefore likely to explain why endometriosis develops in only a subset of women. In addition, invasive diagnostic techniques are not preferred by patients.

The present invention addresses these needs and provides methods of noninvasively detecting and treating endometriosis.

### SUMMARY OF THE INVENTION

A method of non-invasively diagnosing endometriosis in a subject comprising:
passing a sample of menstrual effluent (ME) through (i) a 70 μm pore filter or (ii) a filter that permits through passage of ME single cells but not of ME tissue fragments (i.e. shed endometrial tissue), so as to separate ME tissue fragments from ME single cells;
collecting the ME tissue fragments; treating the ME tissue fragments so as to disaggregate the tissue fragments into cells; performing (i) qPCR and/or digital droplet PCR gene expression analysis or (ii) single cell RNA-sequencing (scRNA-seq) analysis on the cells or (iii) flow cytometry or (iv) other protein analysis; then (1) determining the presence or not of stromal cells exhibiting a phenotype, or gene expression pattern, associated with endometriosis based on results of the qPCR gene expression or scRNA-seq analysis, or protein expression by flow cytometry, and/or
(2) determining levels of (a) uterine NK cells, (b) B cells, and/or (c) T cells based on results of the qPCR gene expression or scRNA-seq analysis or flow cytometry or other protein analysis and determining if the uterine NK cell, B cell, T cell levels are above, below, or within a predetermined control range for uterine NK cell, B cell, T cell levels, respectively; wherein the presence of stromal cells exhibiting a phenotype, or gene or protein expression pattern associated with endometriosis indicates that the sample is from a subject having endometriosis, and/or
a B cell and/or T cell level above the predetermined control range, and a uterine NK cell level below the predetermined control range, indicates that the sample is from a subject having endometriosis.

A method of treating a subject with a dysmenorrhea for endometriosis comprising:
(A) obtaining an identification of the dysmenorrhea in the subject (a) as indicative of endometriosis or (b) as not indicative of endometriosis, wherein identification has been determined by a method comprising:
passing a sample of menstrual effluent (ME) from the subject through (i) a 70 μm pore filter or (ii) a filter that permits through passage of ME single cells but not of ME tissue fragments, so as to separate ME tissue fragments from ME single cells;
collecting the ME tissue fragments;
treating the ME tissue fragments with enzymes so as to disaggregate the tissue fragments into cells, red blood cell lysis and neutrophil depletion;
performing (i) qPCR or digital droplet PCR for gene expression analysis or (ii) scRNA-seq analysis or (iii) or flow cytometry, (iv) or other protein expression analysis on the cells; then
(1) determining the presence or not of stromal cells exhibiting a phenotype, or gene expression pattern, associated with endometriosis based on results of the qPCR gene expression or scRNA-seq analysis, or protein expression by flow cytometry, and/or
(2) determining levels of (a) uterine NK cells, (b) B cells, and/or (c) T cells based on results of the qPCR gene expression or scRNA-seq analysis or flow cytometery or other protein analysis and determining if the uterine NK cell, B cell, T cell levels are above, below, or within a predetermined control range for uterine NK cell, B cell, T cell levels respectively; wherein the presence of stromal cells exhibiting a phenotype, or protein or gene expression pattern, associated with endometriosis indicates that the sample is from a subject having dysmenorrhea indicative of endometriosis, and/or
a B cell and/or T cell level above the predetermined control range, and a uterine NK cell level below the predetermined control range indicates that the sample is from a subject having dysmenorrhea indicative of endometriosis;
and
(B) treating the subject who has been identified as having a dysmenorrhea indicative of endometriosis with an amount of a progestin, a progestin and an estrogen, a danazol, a gonadotropin-releasing hormone agonist, or a birth control pill to the subject effective to treat endometriosis.

A method of determining the efficacy of a treatment for endometriosis comprising:
assessing a baseline level in menstrual effluent of a subject having endometriosis of stromal cells exhibiting a phenotype, or gene expression pattern, associated with endometriosis, and/or levels of (a) uterine NK cells, (b) B cells, and/or (c) T cells based on results of the qPCR gene expression or scRNA-seq analysis or protein expression analysis by any of the methods disclosed herein;
treating the subject by performing a laparoscopic surgery or hysterectomy on the subject, or administering an amount of a progestin, a progestin and an estrogen, a danazol, a gonadotropin-releasing hormone agonist, or a birth control pill to the subject effective to treat endometriosis;
assessing a post-treatment level in menstrual effluent from the subject of stromal cells exhibiting a phenotype, or gene expression pattern, associated with endometriosis, and/or levels of (a) uterine NK cells, (b) B cells, and/or (c) T cells based on results of the qPCR gene expression or scRNA-seq or protein expression analysis;
comparing the post-treatment level with the baseline level of the subject, wherein an improvement in levels of stromal cells exhibiting a phenotype, or gene expression pattern, associated with endometriosis, and/or an improvement in levels of (a) uterine NK cells, (b) B cells, and/or (c) T cells indicates that the treatment is efficacious.

A method of preparing a menstrual effluent (ME) sample for analysis so as to enrich stromal cell content in the sample from 1%, or less, to 10%, or over, comprising: passing the sample of menstrual effluent (ME) through (i) a 70 μm pore filter or (ii) a filter that permits through passage of ME single cells but not of ME tissue fragments, so as to separate ME tissue fragments from ME single cells;
collecting ME tissue fragments that have not passed through the filter;
enzymatically treating fresh or fixed ME tissue fragments so as to disaggregate the tissue fragments into cells; and freezing the cells in a preservative (e.g., methanol or formaldehyde) prior to and/or subsequent to disaggregating the tissue fragments, wherein the preparation results in a stromal cell content in the sample of over 10%.

A kit for non-invasively diagnosing endometriosis in a subject comprising a menstrual effluent (ME) sample; (i) a 70 μm pore filter or (ii) a filter that permits through passage of ME single cells but not of ME tissue fragments; an amount of a collagenase and/or DNase and/or liberase effective to disaggregate an amount of ME tissue fragments.

A method of treating endometriosis in a subject comprising obtaining an identification of the subject as in need of treatment of endometriosis, wherein the subject has been identified as having endometriosis by any of the methods disclosed herein, and treating the subject by performing a laparoscopic surgery or hysterectomy on the subject, or administering an amount of a progestin, a progestin and an estrogen, a danazol, a gonadotropin-releasing hormone agonist, or a birth control pill to the subject effective to treat endometriosis.

Also provided is a method of identifying patients at risk for endometriosis by identifying subclinical inflammation of the uterine lining (e.g., acute or chronic endometritis) by a method comprising passing a sample of menstrual effluent (ME) through (i) a 70 μm pore filter or (ii) a filter that permits through passage of ME single cells but not of ME tissue fragments, so as to separate ME tissue fragments from ME single cells;
collecting the ME tissue fragments; using fresh or fixed tissue fragments;
treating the ME tissue fragments so as to disaggregate the tissue fragments into cells; performing (i) qPCR gene expression analysis or (ii) scRNA-seq analysis or (ii) protein expression analysis on the cells;
then
(1) determining the presence or not of stromal cells exhibiting a phenotype, or gene expression pattern, associated with subclinical inflammation of the uterine lining based on results of the qPCR gene expression or scRNA-seq analysis or protein expression analysis, and/or
(2) determining levels of (a) uterine NK cells, (b) B cells, (c) T cells based on results of the qPCR gene expression or scRNA-seq analysis and determining if the uterine NK cell, B cell, and/or T cell levels are above, below, or within a predetermined control range for uterine NK cell, B cell, and/or T cell levels respectively;
wherein the presence of stromal cells exhibiting a phenotype, or gene expression pattern, associated with subclinical inflammation of the uterine lining indicates that the sample is from a subject having endometriosis, and/or
a B cell and/or T cell level above the predetermined control range, and a uterine NK cell level below the predetermined control range indicates that the sample is from a subject having endometriosis.

### DETAILED DESCRIPTION OF THE INVENTION

A method of non-invasively diagnosing endometriosis in a subject comprising: passing a sample of menstrual effluent (ME) through (i) a 70 μm pore filter or (ii) a filter that permits through passage of ME single cells but not of ME tissue fragments, so as to separate ME tissue fragments from ME single cells;
collecting the ME tissue fragments;
treating the ME tissue fragments so as to disaggregate the tissue fragments into cells;
performing (i) qPCR gene expression analysis or (ii) scRNA-seq analysis or (iii) protein expression analysis on the cells;
then
(1) determining the presence or not of stromal cells exhibiting a phenotype, or gene expression pattern, associated with endometriosis based on results of the qPCR gene expression or scRNA-seq analysis or protein expression analysis, and/or
(2) determining levels of (a) uterine NK cells, (b) B cells, and/or (c) T cells based on results of the qPCR gene expression or scRNA-seq analysis or protein expression analysis and determining if the uterine NK cell, B cell, and/or T cell levels are above, below, or within a predetermined control range for uterine NK cell, B cell, and/or T cell levels respectively;
wherein the presence of stromal cells exhibiting a phenotype, or gene expression pattern, associated with endometriosis indicates that the sample is from a subject having endometriosis, and/or
a B cell and/or T cell level above the predetermined control range, and a uterine NK cell level below the predetermined control range indicates that the sample is from a subject having endometriosis.

In the methods disclosed herein, after collecting the ME tissue fragments, they can be used fresh in subsequent steps or fixed first then used in subsequent steps.

A method of treating a subject with a dysmenorrhea for endometriosis comprising:
(A) obtaining an identification of the dysmenorrhea in the subject (a) as indicative of endometriosis or (b) as not indicative of endometriosis, wherein identification has been determined by a method comprising:
passing a sample of menstrual effluent (ME) from the subject through (i) a 70 μm pore filter or (ii) a filter that permits through passage of ME single cells but not of ME tissue fragments, so as to separate ME tissue fragments from ME single cells;
collecting the ME tissue fragments;
treating the ME tissue fragments so as to disaggregate the tissue fragments into cells;
performing (i) qPCR gene expression analysis or (ii) scRNA-seq analysis on the cells, or (iii) flow cytometry or (iv) other protein expression analysis on the cells; then
(1) determining the presence or not of stromal cells exhibiting a phenotype, or gene expression pattern, associated with endometriosis based on results of the qPCR gene expression or scRNA-seq analysis or flow cytometry or other protein expression analysis, and/or
(2) determining levels of (a) uterine NK cells, (b) B cells, and/or (c) T cells based on results of the qPCR gene expression or scRNA-seq analysis or flow cytometry or other protein expression analysis and determining if the uterine NK cell, B cell, and/or T cell levels are above, below, or within a predetermined control range for uterine NK cell, B cell, and/or T cell levels respectively;
wherein the presence of stromal cells exhibiting a phenotype, or gene expression pattern, associated with endometriosis indicates that the sample is from a subject having dysmenorrhea indicative of endometriosis, and/or
a B cell and/or T cell level above the predetermined control range, and a uterine NK cell level below the predetermined control range indicates that the sample is from a subject having dysmenorrhea indicative of endometriosis;
and
(B) treating the subject who has been identified as having a dysmenorrhea indicative of endometriosis with an amount of a progestin, a progestin and an estrogen, a danazol, a gonadotropin-releasing hormone agonist, an aromatase inhibitor, or a birth control pill to the subject effective to treat endometriosis.

A method of determining the efficacy of a treatment for endometriosis comprising:
assessing a baseline level in menstrual effluent of a subject having endometriosis of stromal cells exhibiting a phenotype, or gene expression pattern, associated with endometriosis, and/or levels of (a) uterine NK cells, (b) B cells, and/or (c) T cells based on results of the qPCR gene expression or scRNA-seq analysis or flow cytometry or other protein expression analysis by the method described herein;
treating the subject by performing a laparoscopic surgery or hysterectomy on the subject, or administering an amount of a progestin, a progestin and an estrogen, a danazol, a gonadotropin-releasing hormone agonist, an aromatase inhibitor, or a birth control pill to the subject effective to treat endometriosis;
assessing a post-treatment level in menstrual effluent from the subject of stromal cells exhibiting a phenotype, or gene expression pattern, associated with endometriosis, and/or levels of (a) uterine NK cells, (b) B cells, and/or (c) T cells based on results of the qPCR gene expression or scRNA-seq analysis or flow cytometry or other protein expression analysis;
comparing the post-treatment level with the baseline level of the subject, wherein an improvement in levels of stromal cells exhibiting a phenotype, or gene expression pattern, associated with endometriosis, and/or an improvement in levels of (a) uterine NK cells, (b) B cells, and/or (c) T cells indicates that the treatment is efficacious. In embodiments, a subject who has been identified as having a dysmenorrhea not indicative of endometriosis is treated for the dysmenorrhea with an amount of a nonsteroidal anti-inflammatory drug. In embodiments, the method further comprises one or more additional iterations of the method so as to determine when treatment can be stopped, wherein when no further improvement is seen in post-treatment levels, then treatment is stopped.

In embodiments, the uterine NK cell, B cell, and/or T cell levels are measured as a fraction or proportion of the relevant cell type as total cells in a sample. In embodiments, the uterine NK cell, B cell, and/or T cell levels are measured as a fraction or proportion of the relevant cell type as total cells in a sample and compared to the respective fraction or proportion of the same cell type in a control sample (e.g. from an otherwise equivalent but non-endometriosis sample.

In embodiments, the methods further comprise enriching the sample for stromal cells by removing CD45+ cells from the sample prior to performing (i) qPCR gene expression analysis or (ii) scRNA-seq analysis. In embodiments, the methods further comprise enriching the sample for stromal cells by removing CD45+ cells from the sample prior to performing flow cytometry or other protein expression analysis.

In embodiments, a subject who has been identified as having a dysmenorrhea not indicative of endometriosis is treated for the dysmenorrhea with an amount of a nonsteroidal anti-inflammatory drug. In embodiments, a subject is identified as having a dysmenorrhea not indicative of endometriosis by (a) having or being diagnosed with a dysmenorrhea but (b) not showing (1) the presence of stromal cells exhibiting a phenotype, or gene expression pattern, associated with endometriosis based on results of the qPCR gene expression or scRNA-seq analysis, or (2) showing uterine NK cell, B cell, and/or T cell levels within a predetermined control range for uterine NK cell, B cell, and/or T cell levels respectively which predetermined control range is not associated with the presence of endometriosis.

In embodiments, the methods further comprise enriching the sample for stromal cells by removing CD45+ cells from the sample prior to performing (i) qPCR gene expression analysis or (ii) scRNA-seq analysis, or (iii) protein analysis

In embodiments, the methods further comprise depleting epithelial cells from the sample prior to performing (i) qPCR gene expression analysis or (ii) scRNA-seq analysis. In embodiments, epithelial cells are removed by a short adhesion step or by using depletion with anti-CD325/EpCAM. In embodiments, the methods further comprise isolating epithelial cells from the sample prior to performing (i) qPCR gene expression analysis, (ii) scRNA-seq analysis or (iii) protein analysis In embodiments, the methods further comprise depleting epithelial cells from the sample prior to performing flow cytometry or other protein expression analysis. flow cytometry or other protein expression analysis.

In embodiments, treating the ME tissue fragments is effected with enzymes so as to disaggregate the tissue fragments into cells. In embodiments, treating the ME tissue fragments further comprises one or more of red blood cell lysis and removing granulocytes. In embodiments, removing granulocytes is effected by CD66b selection, for example via a CD66b cocktail.

In embodiments, the methods can comprise performing (i) qPCR and/or digital droplet PCR gene expression analysis or (ii) single cell RNA-sequencing (scRNA-seq) analysis or (iii) flow cytometry, or (ii) protein expression analysis on the cells.

In embodiments, the method is performed on epithelial cells, myeloid cells, plasma cells, or eosinophils digested from tissue in the ME, mutatis mutandis.

In embodiments, treating the ME tissue fragments so as to disaggregate the tissue fragments into cells comprises contacting the ME tissue fragments with a collagenase. In embodiments, the collagenase is a collagenase I. In further embodiments, the tissue fragments are treated with a DNase and/or liberase. In embodiments, a collagenase I (1 mg/ml)/DNase (0.5 mg/ml)/liberase mixture is used on the tissue for 15 min/37° C. In embodiments, the resultant released cells may be subjected to RBC lysis, neutrophil depletion and/or Ficoll centrifugation to remove dead cells and/or positive immunoselection for specific cell types (stromal cells, T cells, uNK cells, epithelial cells) using antibody coated-magnetic beads before freezing in methanol or other preservative such as formaldehyde, for scRNA seq analysis, qPCR, and/or protein analysis

In embodiments, the methods further comprise freezing the cells using a preservative subsequent or prior to disaggregating the tissue fragments. In embodiments, the cryopreservative comprises methanol or formaldehyde. In embodiments, the methods further comprise freezing the cells in an RNA-stabilizing solution prior to or subsequent to disaggregating the tissue fragments

In embodiments, the methods can further comprise one or more of:
lysing red blood cells in the sample;
depleting neutrophils from the sample; and
removing dead cells from the sample;
prior to performing (i) qPCR gene expression analysis or (ii) scRNA-seq analysis or (iii) protein expression analysis.

In embodiments, the methods further comprise passing the ME tissue fragments separated from the ME single cells through a second filter having a 40 μm pore diameter and wherein the collecting the ME tissue fragments is performed on the ME tissue fragments that do not pass through the second filter.

In embodiments, the sample has been collected in a menstrual cup or a menstrual sponge. In embodiments, the ME sample has previously been collected from a subject.

In embodiments, the methods further comprise separating the stromal, uterine NK cells, B cells, and/or T cells, or tissue-derived epithelial cells, myeloid cells, plasma cells, eosinophils, or other cell types from one another using surface markers prior to performing (i) qPCR or digital droplet PCR gene expression analysis or (ii) scRNA-seq analysis on the cells or (iii) flow cytometry or (iv) other protein expression analysis.

In embodiments, separation is effected using fluorescence-activated cell sorting or magnetic-activated cell sorting. In embodiments, isolation is effected using fluorescence-activated cell sorting or magnetic-activated cell sorting.

In embodiments, the methods comprise determining levels of stromal cells based on results of the qPCR gene expression or scRNA-seq analysis. In embodiments, the methods comprise determining levels of stromal cells based on results of digital droplet PCR gene expression or scRNA-seq analysis or protein expression analysis.

In embodiments, the methods comprise determining the presence or not of stromal cells exhibiting a phenotype, or gene expression pattern, associated with endometriosis based on results of the qPCR gene expression or scRNA-seq analysis, but not determining levels of (a) uterine NK cells, (b) B cells, and/or (c) T cells. In embodiments, the methods comprise determining the presence or not of stromal cells exhibiting a phenotype, or gene expression pattern, or protein expression pattern, associated with endometriosis based on results of the qPCR gene expression or scRNA-seq analysis, but not determining levels of epithelial cells, myeloid cells, plasma cells, or eosinophils digested from tissue in the ME In embodiments, the methods comprise determining levels of (a) uterine NK cells, (b) B cells, and/or (c) T cells based on results of the qPCR gene expression or scRNA-seq analysis and determining if the uterine NK cell, B cell, and/or T cell levels (or other tissue-derived cell types) are above, below, or within a predetermined control range for uterine NK cell, B cell, and/or T cell levels (or other tissue-derived cell types) respectively.

In embodiments, the subject is a human. In embodiments the adult subject is pre-menopausal. In embodiments, the subject is an adolescent. In embodiments, the adolescent is 12 years to <18 years.

In embodiments of the methods, stromal cells are not cultured or maintained in culture prior to digestion and processing. In embodiments, the cells are not cultured prior to analysis. In embodiments, no enzymatic digestion of tissue samples occurs until after filtering to remove single cells.

In embodiments relating to endometriosis, the predetermined control range for B cells, T cells, uterine NK cells, and/or other tissue-derived cell types is determined from one or more ME tissue fragments from one or more control subjects who do not have endometriosis. In embodiments relating to acute or chronic endometritis, the predetermined control range for B cells, T cells and/or uterine NK cells (or other tissue-derived cell types) is determined from one or more ME tissue fragments from one or more control subjects who do not have chronic endometritis.

In embodiments the genes, proteins and/or nucleic acids referred to herein are human.

A method of preparing a menstrual effluent (ME) sample for analysis so as to enrich stromal cell content in the sample from 1%, or less, to 10%, or over, comprising: passing the sample of menstrual effluent (ME) through (i) a 70 μm pore filter or (ii) a filter that permits through passage of ME single cells but not of ME tissue fragments, so as to separate ME tissue fragments from ME single cells;
collecting ME tissue fragments that have not passed through the filter;
enzymatically treating fresh or fixed ME tissue fragments so as to disaggregate the tissue fragments into cells; and
freezing the cells in a preservative (e.g., methanol or formaldehyde) prior to or subsequent to disaggregating the tissue fragments,
wherein the preparation results in a stromal cell content in the sample of over 10%. In embodiments, the method further comprises preparing an ME sample for analysis so as to enrich the stromal cell content in the sample to 20% or more.

In embodiments, the method further comprises preparing an ME sample for analysis so as to enrich the stromal cell content in the sample to 20% or more.

In embodiments, the methods further comprise obtaining the ME sample from the subject.

In embodiments of the methods the stromal cells are stromal fibroblast cells (SFC). In embodiments of the methods the stromal cells are CD45−/CD326−/CD31−/CD 90+/CD105+/CD73+. In embodiments of the methods the stromal cells are CD140b+. In embodiments of the methods the stromal cells exhibit a phenotype, or gene expression pattern, associated with endometriosis, wherein the phenotype or gene expression pattern is a pro-inflammatory or a senescent phenotype or gene expression pattern.

In embodiments, the level of uterine NK cells is determined, and the uterine NK cells are proliferative uterine NK cells.

In embodiments, the proliferative uterine NK cells are positive for human marker of proliferation Ki-67 protein (encoded by MKI67).

In embodiments, the level of proliferative uterine NK cells in an endometriosis subject sample is at least 4-fold lower than in a control sample from a non-endometriosis subject.

In embodiments, the level of proliferative uterine NK cells in an endometriosis subject sample is at least 10-fold lower than in a control sample from a non-endometriosis subject.

In embodiments, the methods further comprise selecting for proliferative uterine NK cells based on expression of a cell proliferation-associated marker. In embodiments, the cell proliferation-associated marker is human marker of proliferation Ki-67, CENPF, UBE2C, ASPM, TOP2A, CKS1B, PCLAF or NUSAP1. In embodiments, the cells other than proliferative uterine NK cells are depleted from the sample by a method comprising negative antibody selection.

In embodiments, determining levels of uterine NK cells is based on results of the qPCR gene expression or scRNA-seq analysis and determining if the expression level of proliferative uterine NK cell human MKI67, or other cell proliferation-associated marker, is above, below, or within a predetermined control range for proliferative uterine NK cell human MKI67, or other cell proliferation-associated marker, respectively.

In embodiments, the presence or not of stromal cells exhibiting a phenotype, or gene expression pattern, associated with endometriosis is determined.

In embodiments, the presence of stromal cells in the sample demonstrating higher level of human matrix Gla protein (MGP), interleukin 11 (IL11) or insulin like growth factor binding protein 1 (IGFBP1) than in a control is indicative of a phenotype, or gene expression pattern, associated with endometriosis.

A kit for non-invasively diagnosing endometriosis in a subject comprising a menstrual effluent (ME) sample; (i) a 70 mm pore filter or (ii) a filter that permits through passage of ME single cells but not of ME tissue fragments; an amount of a collagenase and/or DNase and/or liberase effective to disaggregate an amount of ME tissue fragments. In embodiments, the kit further comprises an amount of preservative. In embodiments, the kit further comprises an amount of an RNA-stabilizing solution. In embodiments, the preservative comprises methanol or formaldehyde

A method of treating endometriosis in a subject comprising obtaining an identification of the subject as in need of treatment of endometriosis, wherein the subject has been identified as having endometriosis by any of the methods of disclosed herein, and treating the subject by performing a laparoscopic surgery t to remove endometriosis lesions, or administering an amount of a progestin, a progestin and an estrogen, a danazol, a gonadotropin-releasing hormone agonist, an aromatase inhibitor, or a birth control pill to the subject in an amount that is effective to treat endometriosis. In embodiments, treatment results in a reduction in one or more of the following symptoms in the subject; chronic pelvic pain, dysmenorrhea, dyspareunia, dysuria, dyschezia, bloating.

In embodiments the laparoscopic surgery is performed to remove ectopic lesions.

Also provided is a method of identifying patients at risk for endometriosis by identifying subclinical inflammation of the uterine lining (e.g. acute or chronic endometritis) by a method comprising passing a sample of menstrual effluent (ME) through (i) a 70 μm pore filter or (ii) a filter that permits through passage of ME single cells but not of ME tissue fragments, so as to separate ME tissue fragments from ME single cells;
collecting the ME tissue fragments; using fresh or fixed tissue fragments; treating the ME tissue fragments so as to disaggregate the tissue fragments into cells; performing (i) qPCR gene expression analysis or (ii) scRNA-seq analysis on the cells or (iii) flow cytometry or (iv) mass spectrometry or other protein analysis on the cells;
then
(1) determining the presence or not of stromal cells exhibiting a phenotype, or gene expression pattern, associated with subclinical inflammation of the uterine lining based on results of the qPCR gene expression or scRNA-seq analysis or flow cytometry or other protein expression analysis, and/or
(2) determining levels of (a) uterine NK cells, (b) B cells, (c) T cells based on results of the qPCR gene expression or scRNA-seq analysis and determining if the uterine NK cell, B cell, and/or T cell levels are above, below, or within a predetermined control range for uterine NK cell, B cell, and/or T cell levels, respectively; wherein the presence of stromal cells exhibiting a phenotype, or gene expression pattern associated with subclinical inflammation of the uterine lining indicates that the sample is from a subject having endometriosis, and/or a B cell and/or T cell level above the predetermined control range, and a uterine NK cell level below the predetermined control range indicates that the sample is from a subject having endometriosis.

The methods herein can be used to identify subjects as at risk for endometriosis. Also provided are methods or treating subjects at risk for endometriosis by administering to the subject locally or systemically an anti-inflammatory agent that targets a cytokine. In embodiments, the cytokines are TNFalpha and/or IL 1beta. Anti-inflammatory agents directed to cytokine(s) are known in the art, including certain organic small molecules (see, e.g. world wide web at ncbi.nlm.nih.gov/pmc/articles/PMC3752337/, incorporated by reference); anticytokines may also be biologics, e.g., monoclonal antibodies or fusion proteins directed against a known cytokine such as TNFalpha or IL-1beta).

Menstrual cups as described herein include, but are not limited to, those sold by Diva International Inc., Ontario, Canada. Sponges for collecting ME as discussed herein include, but are not limited to, polyether polyurethane menstrual sponges

A method of non-invasively diagnosing endometriosis in a subject comprising: passing a sample of menstrual effluent (ME) through (i) a 70 μm pore filter or (ii) a filter that permits through passage of ME single cells but not of ME tissue fragments, so as to separate ME tissue fragments from ME single cells;
collecting the ME tissue fragments; using fresh or fixed tissue fragments;
treating the ME tissue fragments so as to disaggregate the tissue fragments into cells;
performing (i) qPCR gene expression analysis or (ii) scRNA-seq analysis on the cells or (iii) flow cytometry; or (iii) mass spectrometry or other protein analysis on the cells, then determining, based on results of the qPCR gene expression or scRNA-seq analysis or flow cytometry, if the (a) uterine NK cells, (b) B cells, and/or (c) T cells exhibit a gene expression pattern associated with endometriosis,
wherein a gene expression pattern in (a) uterine NK cells, (b) B cells, and/or (c) T cells associated with endometriosis indicates that the sample is from a subject having endometriosis.

As used herein, a predetermined control amount is a value decided or obtained, usually beforehand, as a control. The concept of a control is well-established in the field, and can be determined, in a non-limiting example, empirically from non-afflicted subjects (versus afflicted subjects, including afflicted subjects having different grades of the relevant affliction), and may be normalized as desired (in non-limiting examples, for volume, mass, age, location, gender) to negate the effect of one or more variables.

“And/or” as used herein, for example with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

The inventors have developed new methods of isolating and studying shed eutopic endometrial tissues with abundant stromal cells using ME. Also disclosed are new collection methods of fresh ME that that are practical for both adults and adolescents and can be repeated across menstrual cycles. Importantly, the methods capture the phenotypic state of the eutopic endometrium at a time when ME tissues are delivered into the peritoneal cavity. In addition, disclosed are innovations to rapidly digest endometrial tissue fragments in ME followed by or preceded by, fixation, permeabilization, and/or freezing in a preservative, e.g., in methanol or formaldehyde, so that scRNA-Seq or qPCR or protein analysis (or other sequence analysis methods) can be conveniently and cost-effectively “batched’ and carried out on multiple samples and analyzed simultaneously using, e.g., Demuxlet. This innovation also permits the application of these analyses to compare large numbers of patients and control ME samples, in addition to repeated ME sampling over time to ensure reproducibility. The results provide a full analysis of numerous ME cell types and individual cell subsets, including fresh stromal cells, epithelial cells, uterine NK cells, T cells, B cells, and other cells of the immune system. Interestingly, differences with cultured samples are quite apparent, as described hereinbelow.

Study subjects: Patient recruitment/enrollment has been through the ROSE study which has >1500 participants.

Decidualization defects in ME-eSCs: It has previously been shown that a decidualization defect associated with endometriosis can be readily observed in cultured endometrial stromal cells derived from menstrual effluent (ME-eSCs) (14, 16, 17). In addition, subjects with symptoms of endometriosis without a confirmed diagnosis have a similar defect. Prominent decidualization defects were seen in ME-eSCs (p1) from endometriosis subjects as reflected in production of IGFBP1 by ELISA, comparing cAMP to vehicle after 24 hrs (IGFBP1 ratio). Up to 10% of control subjects have relatively low decidualization capacity). Despite the high accuracy of this observational correlation this approach presents many challenges for developing a diagnostic, since ME-eSCs constitute only 1-3% of the free separated mononuclear cells in fresh ME, and these assays require culturing of these cells. While this is not difficult, but it is very time consuming and expensive, and therefore has limited use as a diagnostic test.

Endometrial tissues are abundant in ME: In the course of processing many samples of ME collected using a menstrual cup, it was observed that clumps of endometrial tissues are present that were not captured by initial approaches. These can be demonstrated by filtering the ME over, e.g., 70 μm filters to enrich for such fragments. Carrying this out on multiple samples it was discovered that intact endometrial tissues can be readily demonstrated by histological analysis of ME. Micrographs of samples showed the presence of fragments of endometrial tissues containing both epithelial and stromal cells, as well as uNK cells. Immunostaining confirmed the presence of endometrial stromal cells (CD10) and uNK cells (CD56) in five individuals.

Several approaches were explored to processing of ‘whole ME’ containing these tissue fragments for scRNA-Seq analysis. Initially, the entire ME sample was digested with collagenase and DNase to release cells from tissue fragments, followed by filtering to capture cells released by digestion, as well the free single cells in the ME. Neutrophils were depleted using magnetic meads and used as a source of genomic DNA for DEMUXLET analysis (neutrophils dominate the cellular content of single cells in ME). This was followed by density gradient separation using Ficoll to remove dead cells. The resulting single cell preparation was frozen in methanol (18) for subsequent single cell processing on the 10× Genomics platform, library construction and scRNA-Seq analysis. Some samples were processed fresh for comparison with methanol freezing.

Before proceeding with detailed analysis of many samples, it was sought to assess any bias that might be induced by methanol freezing of samples by the method of Chen (18). Thus, a comparison of the results from samples with single cell processing of fresh cells was made with processing after freezing in methanol. The results were nearly indistinguishable when comparing 10× genomics processing for scRNA-Seq using methanol-fixed compared to freshly processed ME cells for the scRNA-Seq analysis.

Having the established feasibility of this approach, an scRNA-Seq analysis of ME samples was made, from 11 subjects containing 3 controls, 4 patients with confirmed endometriosis, and 4 samples from patients with symptoms highly suggestive of endometriosis (and impaired decidualization). It was apparent from UMAP plots that multiple distinct clusters of cells can be delineated in these ME samples. This included stromal cells, NK cells, B cells, as well as several subsets of T cells and myeloid cells that cluster towards the center of the plot. A small number epithelial cells (<2-3% of total) were seen also. To understand the distribution of these various clusters comparing the control, endometriosis and symptomatic groups, the results were plotted separately for each group. It was apparent by simple inspection that the number of cells in some clusters are quite different between the clinical groups in particular, uNK cell, B cell, and some T cell subsets. An analysis of odds ratios (OR) for enrichment of these three clusters comparing endo and controls was performed. Both B cells and some T cell subsets are enriched by nearly 4-fold in the ME enriched for enzyme digested tissues of endo cases, while uterine NK (uNK) cells are very highly enriched in the ME of controls, compared with endometriosis cases. It is significant that in prior studies the B cells and T cells were not changed in endometriosis subjects versus control (see, e.g., US 2021-0096137 A1), yet, contrary to this, with the present methods a stark difference was seen in B cells and T cells were not changed in endometriosis subjects versus control.

Also examined were the patterns of gene expression in endometrial stromal cells between endometriosis cases and controls. Strikingly, IGFBP1 mRNA expression is markedly reduced in endometriosis patients compared with controls in the fresh cells. Thus, the data suggest that an analysis of IGFBP1 expression in fresh stromal cells in ME (Table 1) can in fact provide diagnostic power that is similar to the decidualization-induced IGFBP1 protein expression observed in cultured stromal cells following decidualization.

Table 1. Expression of IGFBP1 in fresh ME-derived stromal cells from endometriosis (ENDO) and control (Ctrl) subjects by scRNA-Seq.

| | | | % + | % + | |
|---|---|---|---|---|---|
| Comparison | p_val | avg_log2FC | endo | control | p_val_adj |
| IGFBP1 | 1.55E−15 | −1.79 | 0.2 | 0.4 | 3.35E−11 |
| ENDO vs Ctrl | | | | | |
| IGFBP1 | 4.77E−16 | −1.62 | 0.2 | 0.4 | 1.03E−11 |
| ENDO + | | | | | |
| Sympto vs. Ctrl | | | | | |

In addition, scRNA-Seq permits study subsets of stromal cells and reveal additional gene expression differences between endometriosis patients (cases) and controls scRNA-Seq showed stromal cell subclusters display divergent gene expression patterns comparing controls and endometriosis cases. High expression of both IGFBP1 and LEFTY2 was seen in controls compared to endo in cluster 2, with fold changes in the range of 7-8 (log₂˜2.6-2.8). These transcripts are strongly associated with decidualization. In contrast, cluster 1 was enriched for IL-11 and matrix metalloproteinases in cases with endometriosis.

Alternatively, qPCR may be performed as a transcript gene expression analysis. scRNA-Seq results show a striking increase in both IGFBP1 as well as LEFTY2, a late decidualization marker, in cluster 2 of ME-stromal cells from controls as compared with ME-stromal cells from endo cases. This again strongly suggests that the phenotype of reduced decidualization seen in cultured stromal cells can, surprisingly, in fact be captured in freshly isolated ME-derived endometrial stromal cells from endometriosis cases.

In view of these results, the inventors continued experimenting to find improved methods to enrich ME-endometrial tissues. The results of this scRNA seq analysis were compared to previous reports where free single cells in ME were collected and FACS analysis performed thereupon (14). In this recent approach, specific enrichment and selection for tissue fragments only was performed, filtering out all other free ME cells before tissue digestion and analysis. This is different from the process outlined above where tissue digestion of the entire contents of ME was done, thus including all the free cells in ME, (depleted of neutrophils and RBCs) as well as cells released from digested tissue. A comparison was made of scRNA-Seq of free cells in whole ME with the result after specific tissue fragment enrichment and collection on a 70 μm filter prior to digestion and scRNA-Seq. Analysis of scRNA-Seq results on undigested fresh ME (free single cells) compared with enriched/digested ME-endometrial tissues (right panels) was performed. One subject donated ME using a Diva Cup (Diva International, Inc., Kitchener, Ontario, Canada) (upper panels), the other utilized an external pad collection (lower panels). A dramatic enrichment of both stromal cells and epithelial cells was seen in samples derived from ME that is enriched for enzyme digested endometrial tissues.

A dramatic enrichment of both CD45neg stromal cells and epithelial cells was seen in the processed ME-endometrial tissue samples, compared with a fresh suspension of single cells in ME (without tissues). Interestingly, T cells, B cells and NK cell fractions are present in both types of samples. Of course, the subsets and gene expression patterns in these groups may be different between tissue and ME single cells, but in our view the results of most interest will be found in the cells derived from tissue.

### PUBLICATIONS IN THE FIELD

1. Zondervan K T, Becker C M, Koga K, Missmer S A, Taylor R N and Vigano P. Endometriosis. Nat Rev Dis Primers. 2018; 4:9.30026507
2. Zondervan K T, Becker C M and Missmer S A. Endometriosis. N Engl J Med. 2020; 382:1244-1256.32212520
3. Simoens S, Hummelshoj L and D'Hooghe T. Endometriosis: cost estimates and methodological perspective. HumReprodUpdate. 2007; 13:395-404
4. Marquardt R M, Kim T H, Shin J H and Jeong J W. Progesterone and Estrogen Signaling in the Endometrium: What Goes Wrong in Endometriosis? Int J Mol Sci. 2019; 20.31387263 PMC6695957
5. Brosens I, Brosens J J and Benagiano G. The eutopic endometrium in endometriosis: are the changes of clinical significance? Reprod Biomed Online. 2012; 24:496-502.22417665
6. Liu H and Lang J H. Is abnormal eutopic endometrium the cause of endometriosis? The role of eutopic endometrium in pathogenesis of endometriosis. Med Sci Monit. 2011; 17:RA92-9.21455119 PMC3539524
7. Drury J A, Parkin K L, Coyne L, Giuliani E, Fazleabas A T and Hapangama D K. The dynamic changes in the number of uterine natural killer cells are specific to the eutopic but not to the ectopic endometrium in women and in a baboon model of endometriosis. Reprod Biol Endocrinol. 2018; 16:67.30021652 PMC6052567
8. Chehna-Patel N, Sachdeva G, Gajbhiye R, Warty N and Khole V. “Spot”-ting differences between the ectopic and eutopic endometrium of endometriosis patients. Fertil Steril. 2010; 94:1964-71, 1971 e1.20236630
9. Bulun S E, Cheng Y H, Yin P, Imir G, Utsunomiya H, Attar E, Innes J and Julie Kim J. Progesterone resistance in endometriosis: link to failure to metabolize estradiol. Mol Cell Endocrinol. 2006; 248:94-103.16406281
10. Aghajanova L and Giudice L C. Molecular evidence for differences in endometrium in severe versus mild endometriosis. ReprodSci. 2011; 18:229-251
11. Klemmt P A, Carver J G, Kennedy S H, Koninckx P R and Mardon H J. Stromal cells from endometriotic lesions and endometrium from women with endometriosis have reduced decidualization capacity. FertilSteril. 2006; 85:564-572
12. Sumathi V P and McCluggage W G. CD10 is useful in demonstrating endometrial stroma at ectopic sites and in confirming a diagnosis of endometriosis. J Clin Pathol. 2002; 55:391-2.11986349 PMC1769659
13. Mckinnon B, Mueller M and Montgomery G. Progesterone Resistance in Endometriosis: an Acquired Property? Trends Endocrinol Metab. 2018; 29:535-548.29934050
14. Nayyar A, Saleem M I, Yilmaz M, DeFranco M, Klein G, Elmaliki K M, Kowalsky E, Chatterjee P K, Xue X, Viswanathan R, Shih A J, Gregersen P K and Metz C N. Menstrual Effluent Provides a Novel Diagnostic Window on the Pathogenesis of Endometriosis. Frontiers in Reproductive Health. 2020; 2:1-13
15. Warren L A, Shih A, Renteira S M, Seckin T, Blau B, Simpfendorfer K, Lee A, Metz C N and Gregersen P K. Analysis of menstrual effluent: diagnostic potential for endometriosis. Mol Med. 2018; 24:1.30134794 PMC6016873
16. Szwarc M M, Hai L, Gibbons W E, Peavey M C, White L D, Mo Q, Lonard D M, Kommagani R, Lanz R B, DeMayo F J and Lydon J P. Human endometrial stromal cell decidualization requires transcriptional reprogramming by PLZF. Biol Reprod. 2018; 98:15-27.29186366 PMC5819842
17. Takamoto N, Zhao B, Tsai S Y and DeMayo F J. Identification of Indian hedgehog as a progesterone-responsive gene in the murine uterus. Mol Endocrinol. 2002; 16:2338-48.12351698
18. Gellersen B and Brosens J J. Cyclic decidualization of the human endometrium in reproductive health and failure. EndocrRev. 2014; 35:851-905
19. Shoupe D. The Progestin Revolution: progestins are arising as the dominant players in the tight interlink between contraceptives and bleeding control. Contracept Reprod Med. 2021; 6:3.33517911 PMC7849131
20. Gynecologists TACoOa. Age Related Fertility Decline. Committee Opinion. 2014, reaffirmed 2018; 589:1-3
21. Vitonis A F, Vincent K, Rahmioglu N, Fassbender A, Buck Louis G M, Hummelshoj L, Giudice L C, Stratton P, Adamson G D, Becker C M, Zondervan K T and Missmer S A. World Endometriosis Research Foundation Endometriosis Phenome and Biobanking Harmonization Project: II. Clinical and covariate phenotype data collection in endometriosis research. FertilSteril. 2014; 102:1223-1232
22. van Eijk A M, Zulaika G, Lenchner M, Mason L, Sivakami M, Nyothach E, Unger H, Laserson K and Phillips-Howard P A. Menstrual cup use, leakage, acceptability, safety, and availability: a systematic review and meta-analysis. Lancet Public Health. 2019; 4:e376-e393.31324419 PMC6669309
23. Michalski S A, Chadchan S B, Jungheim E S and Kommagani R. Isolation of Human Endometrial Stromal Cells for In Vitro Decidualization. J Vis Exp. 2018.30222162 PMC6235076
24. Chen J, Cheung F, Shi R, Zhou H, Lu W and Consortium C H I. PBMC fixation and processing for Chromium single-cell RNA sequencing. J Transl Med. 2018; 16:198.30016977 PMC6050658
25. Zhang F, Wei K, Slowikowski K, Fonseka C Y, Rao D A, Kelly S, Goodman S M, Tabechian D, Hughes L B, Salomon-Escoto K, Watts G F M, Jonsson A H, Rangel-Moreno J, Meednu N, Rozo C, Apruzzese W, Eisenhaure T M, Lieb D J, Boyle D L, Mandelin A M, 2nd, Accelerating Medicines Partnership Rheumatoid A, Systemic Lupus Erythematosus C, Boyce B F, DiCarlo E, Gravallese E M, Gregersen P K, Moreland L, Firestein G S, Hacohen N, Nusbaum C, Lederer J A, Perlman H, Pitzalis C, Filer A, Holers V M, Bykerk V P, Donlin L T, Anolik J H, Brenner M B and Raychaudhuri S. Defining inflammatory cell states in rheumatoid arthritis joint synovial tissues by integrating single-cell transcriptomics and mass cytometry. Nat Immunol. 2019; 20:928-942.31061532 PMC6602051
26. Yu J, Berga S L, Zou W and Taylor R N. Interleukin-1beta inhibits estrogen receptor-alpha, progesterone receptors A and B and biomarkers of human endometrial stromal cell differentiation: implications for endometriosis. Mol Hum Reprod. 2019; 25:625-637.31408162 PMC6821275
27. Hall O J and Klein S L. Progesterone-based compounds affect immune responses and susceptibility to infections at diverse mucosal sites. Mucosal Immunol. 2017; 10:1097-1107.28401937
28. Scarpin K M, Graham J D, Mote P A and Clarke C L. Progesterone action in human tissues: regulation by progesterone receptor (PR) isoform expression, nuclear positioning and coregulator expression. Nucl Recept Signal. 2009; 7:e009.20087430 PMC2807635
29. Sitruk-Ware R. New progestagens for contraceptive use. Hum Reprod Update. 2006; 12:169-78.16291771
30. Reis F M, Coutinho L M, Vannuccini S, Batteux F, Chapron C and Petraglia F. Progesterone receptor ligands for the treatment of endometriosis: the mechanisms behind therapeutic success and failure. Hum Reprod Update. 2020; 26:565-585.32412587 PMC7317284
31. Bedaiwy M A, Dahoud W, Skomorovska-Prokvolit Y, Yi L, Liu J H, Falcone T, Hurd W W and Mesiano S. Abundance and Localization of Progesterone Receptor Isoforms in Endometrium in Women With and Without Endometriosis and in Peritoneal and Ovarian Endometriotic Implants. Reprod Sci. 2015; 22:1153-61.26037298 PMC5933169
32. Gronemeyer H, Meyer M E, Bocquel M T, Kastner P, Turcotte B and Chambon P. Progestin receptors: isoforms and antihormone action. J Steroid Biochem Mol Biol. 1991; 40:271-8.1958531
33. Kastner P, Krust A, Turcotte B, Stropp U, Tora L, Gronemeyer H and Chambon P. Two distinct estrogen-regulated promoters generate transcripts encoding the two functionally different human progesterone receptor forms A and B. EMBO J. 1990; 9:1603-14.2328727 PMC551856
34. Giangrande P H, Kimbrel E A, Edwards D P and McDonnell D P. The opposing transcriptional activities of the two isoforms of the human progesterone receptor are due to differential cofactor binding. Mol Cell Biol. 2000; 20:3102-15.10757795 PMC85605
35. Lydon J P, DeMayo F J, Funk C R, Mani S K, Hughes A R, Montgomery C A, Jr., Shyamala G, Conneely O M and O'Malley B W. Mice lacking progesterone receptor exhibit pleiotropic reproductive abnormalities. Genes Dev. 1995; 9:2266-78.7557380
36. Mulac-Jericevic B, Mullinax R A, DeMayo F J, Lydon J P and Conneely O M. Subgroup of reproductive functions of progesterone mediated by progesterone receptor-B isoform. Science. 2000; 289:1751-4.10976068
37. Patel B G, Rudnicki M, Yu J, Shu Y and Taylor R N. Progesterone resistance in endometriosis: origins, consequences and interventions. Acta Obstet Gynecol Scand. 2017; 96:623-632.28423456
38. Bergqvist A, Ljungberg O and Skoog L. Immunohistochemical analysis of oestrogen and progesterone receptors in endometriotic tissue and endometrium. Hum Reprod. 1993; 8: 1915-22.8288760
39. Igarashi T M, Bruner-Tran K L, Yeaman G R, Lessey B A, Edwards D P, Eisenberg E and Osteen K G. Reduced expression of progesterone receptor-B in the endometrium of women with endometriosis and incocultures of endometrial cells exposed to 2,3,7,8-tetrachlorodibenzo-p-dioxin. Fertil Steril. 2005; 84:67-74.16009159
40. Gentilini D, Vigano P, Vignali M, Busacca M, Panina-Bordignon P, Caporizzo E and Di Blasio A M. Endometrial stromal progesterone receptor-A/progesterone receptor-B ratio: no difference between women with and without endometriosis. Fertil Steril. 2010; 94:1538-1540.20097334
41. Nisolle M, Casanas-Roux F, Wyns C, de Menten Y, Mathieu P E and Donnez J. Immunohistochemical analysis of estrogen and progesterone receptors in endometrium and peritoneal endometriosis: a new quantitative method. Fertil Steril. 1994; 62:751-9.7523199
42. Storer C L, Dickey C A, Galigniana M D, Rein T and Cox M B. FKBP51 and FKBP52 in signaling and disease. Trends Endocrinol Metab. 2011; 22:481-90.21889356 PMC3229651
43. Tranguch S, Cheung-Flynn J, Daikoku T, Prapapanich V, Cox M B, Xie H, Wang H, Das S K, Smith D F and Dey S K. Cochaperone immunophilin FKBP52 is critical to uterine receptivity for embryo implantation. Proc Natl Acad Sci USA. 2005; 102:14326-31.16176985 PMC1242310
44. Tranguch S, Wang H, Daikoku T, Xie H, Smith D F and Dey S K. FKBP52 deficiency-conferred uterine progesterone resistance is genetic background and pregnancy stage specific. J Clin Invest. 2007; 117: 1824-34.17571166 PMC1888571
45. Hirota Y, Tranguch S, Daikoku T, Hasegawa A, Osuga Y, Taketani Y and Dey S K. Deficiency of immunophilin FKBP52 promotes endometriosis. Am J Pathol. 2008; 173:1747-57.18988805 PMC2626386
46. Maguire O, Collins C, O'Loughlin K, Miecznikowski J and Minderman H. Quantifying nuclear p65 as a parameter for NF-kappaB activation: Correlation between ImageStream cytometry, microscopy, and Western blot. Cytometry A. 2011; 79:461-9.21520400 PMC3140714
47. Stone R C, Feng D, Deng J, Singh S, Yang L, Fitzgerald-Bocarsly P, Eloranta M L, Ronnblom L and Barnes B J. Interferon regulatory factor 5 activation in monocytes of systemic lupus erythematosus patients is triggered by circulating autoantigens independent of type I interferons. Arthritis Rheum. 2012; 64:788-98.21968701 PMC3288585
48. Reyes M, Billman K, Hacohen N and Blainey P C. Simultaneous profiling of gene expression and chromatin accessibility in single cells. Adv Biosyst. 2019; 3.31853478 PMC6919553
49. Skene P J and Henikoff S. An efficient targeted nuclease strategy for high-resolution mapping of DNA binding sites. Elife. 2017; 6.28079019 PMC5310842
50. Meers M P, Bryson T D, Henikoff J G and Henikoff S. Improved CUT&RUN chromatin profiling tools. Elife. 2019; 8.31232687 PMC6598765
51. Zuba-Surma E K, Kucia M, Abdel-Latif A, Lillard J W, Jr. and Ratajczak M Z. The ImageStream System: a key step to a new era in imaging. Folia Histochem Cytobiol. 2007; 45:279-90.18165167
52. Banga J, Srinivasan D, Sun C C, Thompson C D, Milletti F, Huang K S, Hamilton S, Song S, Hoffman A F, Qin Y G, Matta B, LaPan M, Guo Q, Lu G, Li D, Qian H, Bolin D R, Liang L, Wartchow C, Qiu J, Downing M, Narula S, Fotouhi N, DeMartino J A, Tan S L, Chen G and Barnes B J. Inhibition of IRF5 cellular activity with cell-penetrating peptides that target homodimerization. Sci Adv. 2020; 6:eaay 1057.32440537 PMC7228753
53. De S, Zhang B, Shih T, Singh S, Winkler A, Donnelly R and Barnes B J. B Cell-Intrinsic Role for IRF5 in TLR9/BCR-Induced Human B Cell Activation, Proliferation, and Plasmablast Differentiation. Front Immunol. 2017; 8:1938.29367853 PMC5768180
54. Li D, Matta B, Song S, Nelson V, Diggins K, Simpfendorfer K R, Gregersen P K, Linsley P and Barnes B J. IRF5 genetic risk variants drive myeloid-specific IRF5 hyperactivation and presymptomatic SLE. JCI Insight. 2020; 5.31877114 PMC7098722
55. Song S, De S, Nelson V, Chopra S, LaPan M, Kampta K, Sun S, He M, Thompson C D, Li D, Shih T, Tan N, Al-Abed Y, Capitle E, Aranow C, Mackay M, Clapp W L and Barnes B J. Inhibition of IRF5 hyperactivation protects from lupus onset and severity. J Clin Invest. 2020; 130:6700-6717.32897883 PMC7685739
56. Winkler A, Sun W, De S, Jiao A, Nusrat Sharif M, Symanowicz P T, Athale S, Shin J H, Wang J, Jacobson B A, Ramsey S J, Dower K, Andreyeva T, Liu H, Hegen M, Homer B L, Brodfuehrer J, Tilley M, Gilbert S A, Danto S I, Beebe J J, Barnes B J, Pascual V, Lin L L, Kilty I, Fleming M and Rao V R. The IRAK4 kinase inhibitor PF-06650833 blocks inflammation in preclinical models of rheumatologic disease and in humans enrolled in a randomized clinical trial. Arthritis Rheumatol. 2021.34423919
57. Buenrostro J D, Wu B, Chang H Y and Greenleaf W J. ATAC-seq: A Method for Assaying Chromatin Accessibility Genome-Wide. Curr Protoc Mol Biol. 2015; 109:21 29 1-9.25559105 PMC4374986
58. Buenrostro J D, Giresi P G, Zaba L C, Chang H Y and Greenleaf W J. Transposition of native chromatin for fast and sensitive epigenomic profiling of open chromatin, DNA-binding proteins and nucleosome position. Nat Methods. 2013; 10:1213-8.24097267 PMC3959825
59. Taylor R N, Kane M A and Sidell N. Pathogenesis of Endometriosis: Roles of Retinoids and Inflammatory Pathways. Semin Reprod Med. 2015; 33:246-56.26132929 PMC4745121
60. Chantalat E, Valera M C, Vaysse C, Noirrit E, Rusidze M, Weyl A, Vergriete K, Buscail E, Lluel P, Fontaine C, Arnal J F and Lenfant F. Estrogen Receptors and Endometriosis. Int J Mol Sci. 2020; 21.32316608 PMC7215544
61. Park S, Lim W, Bazer F W, Whang K Y and Song G. Quercetin inhibits proliferation of endometriosis regulating cyclin DI and its target microRNAs in vitro and in vivo. J Nutr Biochem. 2019; 63:87-100.30359864
62. Jamali N, Zal F, Mostafavi-Pour Z, Samare-Najaf M, Poordast T and Dehghanian A. Ameliorative Effects of Quercetin and Metformin and Their Combination Against Experimental Endometriosis in Rats. Reprod Sci. 2021; 28:683-692.33141412
63. Patel B, Elguero S, Thakore S, Dahoud W, Bedaiwy M and Mesiano S. Role of nuclear progesterone receptor isoforms in uterine pathophysiology. Hum Reprod Update. 2015; 21:155-73.25406186 PMC4366574
64. Ahn S H, Edwards A K, Singh S S, Young S L, Lessey B A and Tayade C. IL-17A Contributes to the Pathogenesis of Endometriosis by Triggering Proinflammatory Cytokines and Angiogenic Growth Factors. J Immunol. 2015; 195:2591-600.26259585 PMC4561197
65. Maia H, Jr., Haddad C, Coelho G and Casoy J. Role of inflammation and aromatase expression in the eutopic endometrium and its relationship with the development of endometriosis. Womens Health (Lond). 2012; 8:647-58.23181530
66. Matteo M, Cicinelli E, Neri M, Carrubba R, Carpagnano F A, Romeo F, Scutiero G, Greco P, Garlanda C, Vendemiale G, Levi Setti P E and Serviddio G. Pro-inflammatory M1/Th1 type immune network and increased expression of TSG-6 in the eutopic endometrium from women with endometriosis. Eur J Obstet Gynecol Reprod Biol. 2017; 218:99-105.28963923
67. Vallve-Juanico J. Houshdaran S and Giudice L C. The endometrial immune environment of women with endometriosis. Hum Reprod Update. 2019; 25:564-591.31424502 PMC6737540
68. Grandi G, Mueller M, Bersinger N A, Cagnacci A, Volpe A and Mckinnon B. Does dienogest influence the inflammatory response of endometriotic cells? A systematic review: Inflamm Res. 2016; 65:183-92.26650031
69. Cakmak H, Seval-Celik Y, Arlier S, Guzeloglu-Kayisli O, Schatz F, Arici A and Kayisli U A. p38 Mitogen-Activated Protein Kinase is Involved in the Pathogenesis of Endometriosis by Modulating Inflammation, but not Cell Survival. Reprod Sci. 2018; 25:587-597.28845752
70. Payan-Carreira R, Pires M A, Strom Holst B and Rodriguez-Martinez H. Tumour necrosis factor in the canine endometrium: an immunohistochemical study. Reprod Domest Anim. 2011; 46:410-8.20880318
71. Tabibzadeh S. Ubiquitous expression of TNF-alpha/cachectin immunoreactivity in human endometrium. Am J Reprod Immunol. 1991; 26:1-4.1741931
72. Hunt J S, Chen H L, Hu X L and Tabibzadeh S. Tumor necrosis factor-alpha messenger ribonucleic acid and protein in human endometrium. Biol Reprod. 1992; 47:141-7.1637942
73. Okuda K, Sakumoto R, Okamoto N, Acosta T J, Abe H, Okada H, Sinowatz F and Skarzynski D J. Cellular localization of genes and proteins for tumor necrosis factor-alpha (TNF), TNF receptor types I and II in bovine endometrium. Mol Cell Endocrinol. 2010; 330:41-8.20705117
74. Cicinelli E, Trojano G, Mastromauro M, Vimercati A, Marinaccio M, Mitola P C, Resta L and de Ziegler D. Higher prevalence of chronic endometritis in women with endometriosis: a possible etiopathogenetic link. Fertil Steril. 2017; 108:289-295 e1.28624114
75. Takebayashi A, Kimura F, Kishi Y, Ishida M, Takahashi A, Yamanaka A, Takahashi K, Suginami H and Murakami T. The association between endometriosis and chronic endometritis. PLOS One. 2014; 9:e88354.24558386 PMC3928198
76. Tortorella C, Piazzolla G, Matteo M, Pinto V, Tinelli R, Sabba C, Fanelli M and Cicinelli E. Interleukin-6, interleukin-1beta, and tumor necrosis factor alpha in menstrual effluents as biomarkers of chronic endometritis. Fertil Steril. 2014; 101:242-7.24314919
77. Wu D, Kimura F, Zheng L, Ishida M, Niwa Y, Hirata K, Takebayashi A, Takashima A, Takahashi K, Kushima R, Zhang G and Murakami T. Chronic endometritis modifies decidualization in human endometrial stromal cells. Reprod Biol Endocrinol. 2017; 15:16.28259137 PMC5336610
78. Barragan F, Irwin J C, Balayan S, Erikson D W, Chen J C, Houshdaran S, Piltonen T T, Spitzer T L, George A, Rabban J T, Nezhat C and Giudice L C. Human Endometrial Fibroblasts Derived from Mesenchymal Progenitors Inherit Progesterone Resistance and Acquire an Inflammatory Phenotype in the Endometrial Niche in Endometriosis. Biol Reprod. 2016; 94: 118.27075616 PMC4939744
79. Malvezzi H, Hernandes C, Piccinato C and Podgaec S. Interleukin in endometriosis-associated infertility-pelvic pain: systematic review and meta-analysis. Reproduction. 2019.30933927
80. Machairiotis N, Vasilakaki S and Thomakos N. Inflammatory Mediators and Pain in Endometriosis: A Systematic Review. Biomedicines. 2021; 9.33435569 PMC7826862
81. D'Hooghe T M, Nugent N P, Cuneo S, Chai D C, Deer F, Debrock S, Kyama C M, Mihalyi A and Mwenda J M. Recombinant human TNFRSF1A (r-hTBP1) inhibits the development of endometriosis in baboons: a prospective, randomized, placebo- and drug-controlled study. Biol Reprod. 2006; 74:131-6.16177224
82. Barrier B F, Bates G W, Leland M M, Leach D A, Robinson R D and Propst A M. Efficacy of anti-tumor necrosis factor therapy in the treatment of spontaneous endometriosis in baboons. Fertil Steril. 2004; 81 Suppl 1:775-9.15019808
83. Koninckx P R, Craessaerts M, Timmerman D, Cornillie F and Kennedy S. Anti-TNF-alpha treatment for deep endometriosis-associated pain: a randomized placebo-controlled trial. Hum Reprod. 2008; 23:2017-23.18556683 PMC2517154
84. Sikora J, Mielczarek-Palacz A and Kondera-Anasz Z. Association of the Precursor of Interleukin-1beta and Peritoneal Inflammation-Role in Pathogenesis of Endometriosis. J Clin Lab Anal. 2016; 30:831-837.27018977 PMC6807114

1. A method of non-invasively diagnosing endometriosis in a subject comprising:
passing a sample of menstrual effluent (ME) through (i) a 70 μm pore filter or (ii) a filter that permits through passage of ME single cells but not of ME tissue fragments, so as to separate ME tissue fragments from ME single cells;
collecting the ME tissue fragments;
treating the ME tissue fragments so as to disaggregate the tissue fragments into cells;
performing (i) qPCR and/or digital droplet PCR gene expression analysis or (ii) single cell RNA-sequencing (scRNA-seq) analysis or (iii) flow cytometry (iv) or protein expression analysis on the cells;
(1) determining, based on results of the qPCR or digital droplet PCR gene expression or scRNA-seq analysis or flow cytometry or protein expression analysis, the presence or not of stromal cells or epithelial cells exhibiting a phenotype, or gene expression pattern, associated with endometriosis, and/or
(2) determining levels of (a) uterine NK cells, (b) B cells, and/or (c) T cells based on results of the qPCR gene expression or scRNA-seq analysis or flow cytometry or protein expression analysis and determining if the uterine NK cell, B cell, and/or T cell levels are above, below, or within a predetermined control range for uterine NK cell, B cell, and/or T cell levels respectively;
wherein the presence of stromal cells exhibiting a phenotype, or gene expression pattern or protein expression pattern, associated with endometriosis indicates that the sample is from a subject having endometriosis, and/or
a B cell and/or T cell level above the predetermined control range, and a uterine NK cell level below the predetermined control range indicates that the sample is from a subject having endometriosis.

2. A method of treating a subject with a dysmenorrhea for endometriosis comprising:
(A) obtaining an identification of the dysmenorrhea in the subject (a) as indicative of endometriosis or (b) as not indicative of endometriosis, wherein identification has been determined by a method comprising:
passing a sample of menstrual effluent (ME) from the subject through a 70 μm pore filter or a filter that permits through passage of ME single cells but not of ME tissue fragments, so as to separate ME tissue fragments from ME single cells;
collecting the ME tissue fragments;
treating the ME tissue fragments so as to disaggregate the tissue fragments into cells;
performing (i) qPCR gene expression analysis or (ii) scRNA-seq analysis or (iii) flow cytometry or (iv) mass spectrometry or other protein analysis on the cells.
then
(1) determining the presence or not of stromal cells exhibiting a phenotype, or gene expression pattern, associated with endometriosis based on results of the qPCR gene expression or scRNA-seq analysis or flow cytometry, and/or
(2) determining levels of (a) uterine NK cells, (b) B cells, and/or (c) T cells based on results of the qPCR gene expression or scRNA-seq analysis and determining if the uterine NK cell, B cell, and/or T cell levels are above, below, or within a predetermined control range for uterine NK cell, B cell, and/or T cell levels respectively;
wherein the presence of stromal cells exhibiting a phenotype, or gene expression pattern, associated with endometriosis indicates that the sample is from a subject having dysmenorrhea indicative of endometriosis, and/or
a B cell and/or T cell level above the predetermined control range, and a uterine NK cell level below the predetermined control range indicates that the sample is from a subject having dysmenorrhea indicative of endometriosis; and
(B) treating the subject who has been identified as having a dysmenorrhea indicative of endometriosis with an amount of a progestin, a progestin and an estrogen, a danazol, a gonadotropin-releasing hormone agonist, an aromatase inhibitor, or a birth control pill to the subject effective to treat endometriosis.

3. The method of claim 2, wherein a subject who has been identified as having a dysmenorrhea not indicative of endometriosis is treated for the dysmenorrhea with an amount of a nonsteroidal anti-inflammatory drug or other anti-inflammatory drug.

4. The method of claim 2, further comprising enriching the sample for stromal cells by removing CD45+ cells from the sample prior to performing (i) qPCR gene expression analysis or (ii) scRNA-seq analysis or (iii) flow cytometry, or (iv) mass spectrometry or other protein analysis.

5. The method of claim 2, further comprising depleting epithelial cells from the sample prior to performing (i) qPCR gene expression analysis or (ii) scRNA-seq analysis or (iii) flow cytometry, or (iv) mass spectrometry or other protein analysis.

6. The method of claim 2, wherein treating the ME tissue fragments so as to disaggregate the tissue fragments into cells comprises contacting the ME tissue fragments with a collagenase, DNAse and/or liberase.

7. The method of claim 2, further comprising freezing and or storing the cells in a preservative, or RNA-stabilizing solution, prior to, or subsequent to disaggregating the tissue fragments.

8. The method of claim 2, further comprising one or more of:
lysing red blood cells in the sample;
depleting neutrophils from the sample; and
removing dead cells from the sample;
prior to performing (i) qPCR gene expression analysis or (ii) scRNA-seq analysis or (iii) flow cytometry, or (iv) mass spectrometry or other protein expression analysis.

9. The method of claim 2, further comprising passing the ME tissue fragments separated from the ME single cells through a second filter having a 40 μm pore diameter and wherein the collecting the ME tissue fragments is performed on the ME tissue fragments that do not pass through the second filter.

10. The method of claim 2, wherein the sample has been collected in a menstrual cup or a menstrual sponge.

11. The method of claim 2, further comprising separating the stromal, uterine NK cells, B cells, and/or T cells from one another using surface markers prior to performing (i) qPCR gene expression analysis or (ii) scRNA-seq analysis or (iii) flow cytometry, or (iv) mass spectrometry or other protein analysis on the cells.

12. The method of claim 11, wherein separation and/or isolation is effected using fluorescence-activated cell sorting or magnetic-activated cell sorting.

13. The method of claim 2, comprising determining levels of stromal cells based on results of the qPCR gene expression or scRNA-seq analysis.

14. The method of claim 2, comprising determining the presence or not of stromal cells exhibiting a phenotype, or gene expression pattern, associated with endometriosis based on results of the qPCR gene expression or scRNA-seq analysis, but not determining levels of (a) uterine NK cells, (b) B cells, and/or (c) T cells.

15. The method of claim 2, comprising determining levels of (a) uterine NK cells, (b) B cells, and/or (c) T cells based on results of the qPCR gene expression or scRNA-seq analysis and determining if the uterine NK cell, B cell, and/or T cell levels are above, below, or within a predetermined control range for uterine NK cell, B cell, and/or T cell levels respectively.

16. The method of claim 2, wherein the subject is a human.

17. The method of claim 16, wherein the subject is an adolescent.

18-20. (canceled)

21. A method of preparing a menstrual effluent (ME) sample for analysis so as to enrich stromal cell content in the sample from 3%, or less, to 10%, or over, comprising:
passing the sample of menstrual effluent (ME) through (i) a 70 μm pore filter or (ii) a filter that permits through passage of ME single cells but not of ME tissue fragments, so as to separate ME tissue fragments from ME single cells;
collecting ME tissue fragments that have not passed through the filter;
enzymatically treating the ME tissue fragments so as to disaggregate the tissue fragments into cells; and fixation, permeabilization and/or freezing the cells in methanol and/or other preservatives, or RNA-stabilizing solution, prior to or subsequent to disaggregating the tissue fragments, wherein the preparation results in a stromal cell content in the sample of over 10%.

22. The method of claim 21, comprising preparing an ME sample for analysis so as to enrich the stromal cell content in the sample to 20% or more.

23-39. (canceled)

40. A method of treating endometriosis in a subject comprising obtaining an identification of the subject as in need of treatment of endometriosis, wherein the subject has been identified as having endometriosis by the method of claim 1, and treating the subject by performing a laparoscopic surgery or hysterectomy on the subject, or administering an amount of a progestin, a progestin and an estrogen, a danazol, a gonadotropin-releasing hormone agonist, an aromatase inhibitor, or a birth control pill to the subject effective to treat endometriosis.

41. (canceled)
